(19) United States
(12) Patent Application Publication
(10) Pub. No.: US 2021/0350934 A1
(43) Pub. Date: Nov. 11, 2021

(54) SYNTHETIC TUMOR MODELS FOR USE IN THERAPEUTIC RESPONSE PREDICTION

(71) Applicant: QUANTITATIVE IMAGING SOLUTIONS, LLC, Newton, MA (US)

(72) Inventors: Charles Matthew Kinsey, Willison, VT (US); Raul San Jose Estepar, Wellesley, MA (US); George Washko, West Roxbury, MA (US)

(21) Appl. No.: 17/313,525

(22) Filed: May 6, 2021

Related U.S. Application Data
(60) Provisional application No. 63/020,771, filed on May 6, 2020.

(51) Int. Cl. G16H 50/50; G06T 11/00; G06K 9/62; G06F 30/23; G06F 30/27; G16H 50/70; G16H 30/20; G16H 30/40; G16H 50/20; G16H 20/10; G16H 20/30; G16H 70/60

(52) U.S. Cl. CPC G16H 50/50; G06F 2111/10; G06K 9/6256; G06K 9/6215; G06F 30/23; G06F 30/27; G16H 50/70; G16H 30/20; G16H 30/40; G16H 50/20; G16H 20/10; G16H 20/30; G16H 70/60; G06K 2209/05; G06T 11/006

(57) ABSTRACT

Systems and methods for creating therapeutic response predictions based on a synthetic tumor model generated based on micro-scale data associated with one or more parameters representative of one or more biological characteristics of tumors, where synthetic CT images constructed via back projection of the synthetic tumor model and distribution/response of one or more therapeutic therapies predicted for the synthetic tumor model are used to train an unsupervised learning model for determining a personalized treatment plan for a patient's tumor.

### CROSS-REFERENCE TO RELATED APPLICATIONS

This application claims the benefit of U.S. provisional application No. 63/020,771, filed on May 6, 2020, which is expressly incorporated by reference herein in its entirety.

### TECHNICAL FIELD

The subject matter of this disclosure relates in general to the field of creating synthetic models of tumors and obtaining associated therapeutic response predictions therewith.

### BACKGROUND

The recent development of immunotherapy for the treatment of cancers has resulted in dramatic improvements in survival. However only a minority of patients respond to these therapies. For instance, one of the goals of intravenous (“IV”) immunotherapy is to activate CD8+ T lymphocytes (“CTLs”) to kill within the tumor micro-environment. However, unlike a cytotoxic agent like cisplatin, the maximum tolerated dose of an immunotherapy drug may not result in the optimal treatment effect. Furthermore, a local concentration of an IV agent within a tumor is also highly variable, resulting in different responses in different regions of the tumor.

Given such variance, there has not been a readily applicable system to predict the distribution or local response. The problem with the lack of such a system for predicting the distribution of the IV agent or the local response is further compounded by the development of techniques to deliver agents directly into the tumor (e.g., intratumoral (“IT”) administration) and the recognition of the importance of multimodal or combination of therapies.

In addition, while modalities such as CT scans are well-known for their use in determining the shape or morphology of a tumor, known approaches and modern imaging techniques are ineffective in resolving intratumoral features such as blood vessel location and micro-vessel density. Similarly, the cellular components within a tumor are at best identifiable using modern CT scans in terms of variations in CT density (e.g. Hounsfield unit). However, there are no known and effective approaches for linking specific cellular and stromal features to the associated CT density characteristics, due to, for example, the inability of known approaches to accurately determine cellular composition in vivo. Moreover, evaluating tumors that have been resected is limited by the lack of blood flow, where blood flow is an important contributor to density and is further complicated by the effects of tissue desiccation.

And while unsupervised machine learning techniques, such as deep learning, are changing the landscape of personalized medicine through the capability of predicting disease state and therapeutic response, these techniques rely on large numbers of exemplars to train a neural network, which may then be used for predicting the disease or therapeutic response in a new population. Yet, the number of exemplars needed to accurately train a deep learning model can be in the millions, which is orders of magnitude larger than typical medical data sets.

Therefore, there is a need in the art for a system and method for generating synthetic tumor models as well as therapeutic response prediction models using in silico systems and methods based on synthetic tumor models to more accurately predict and correlate therapeutic responses, with accuracy lent from deep learning models.

### SUMMARY

To address the above-noted problems, systems and methods are disclosed herein for generating synthetic tumor models as well as therapeutic response prediction models based on the synthetic tumor models. According to some examples, the method includes generating micro-scale data for the synthetic tumor model, generated micro-scale data associated with one or more parameters representative of one or more biological characteristics of tumors, the micro-scale data comprising randomized variations within known respective biological ranges of each of the one or more parameters.

The micro-scale data may be randomized based on one or more parameters including at least one of a tumor morphology parameter, a tumor oxygen intake parameter, a tumor cellular components parameter, and a perinodular density components parameter. The tumor morphology parameter may depend on a principal component analysis weight for a PCA model of nodule segmentation based on CT scans of in vivo tumors. The tumor oxygen intake parameter may depend on a vascular generator that varies tumor vascularity based on cellular oxygen consumption described by an oxygenation map formed by a tree growth algorithm. The tumor cellular components parameter may depend on ratios of cellular tissue constituents per unit volume. The perinodular density components parameter may depend on ratios of perinodular cellular tissue constituents per unit volume. According to some examples, the method includes forming the synthetic tumor model based on the generated micro-scale data.

According to some examples, the method includes constructing one or more synthetic CT images via back projection of the synthetic tumor model. The one or more synthetic CT images may be based on an image-based therapeutic response model fitting that is trained by other CT images associated with corresponding therapeutic responses. In another example of the constructing one or more synthetic CT images, the method may comprise partial volume averaging of density values at a resolution of a CT grid by at least one of using a Gaussian kernel that simulates effects of a reconstruction kernel and introducing Gaussian noise that simulates quantum noise at a CT detector. In another example of the constructing one or more synthetic CT images, the method may comprises incorporating model weights with the one or more synthetic CT images to condition a therapeutic response model for extrapolation of personalized therapeutic responses based on in-vivo subject CT scans.

According to some examples, the method includes using a generative adversarial network that uses a deep-learning algorithm to add structural and functional details captured from CT images of actual tumors to create one or more improved synthetic CT back projections. According to some examples, the method includes training, based on the one or more improved synthetic CT back projections and associated tumor FEMs, an unsupervised learning model to distinguish between CT images of synthetic tumor models associated with responsive outcomes versus CT images of synthetic tumor models associated with less responsive outcomes. The unsupervised learning model may be a non-linear regression model encoded as a deep neural network or regression method.

According to some examples, the method includes determining, based on the unsupervised learning model, a personalized treatment plan based on similar characteristics found in the one or more improved synthetic CT back projections and a CT scan a patient tumor to determine at least one an optimal location of placement of a delivery device within the patient tumor and a distribution of a drug, energy, or other intratumoral therapeutic modality or a combination thereof within the patient tumor.

### BRIEF DESCRIPTION OF THE DRAWINGS

In order to describe the manner in which the above-recited and other advantages and features of the disclosure can be obtained, a more particular description of the principles briefly described above will be rendered by reference to specific embodiments thereof which are illustrated in the appended drawings. Understanding that these drawings depict only exemplary embodiments of the disclosure and are not therefore to be considered to be limiting of its scope, the principles herein are described and explained with additional specificity and detail through the use of the accompanying drawings in which:

FIG. 1A illustrates a high level overview of functional blocks used in the creation of an in silico system for modeling tumors and the use thereof therapeutic response prediction, according to example aspects of this disclosure;

FIGS. 1B-1 and 1B-2 illustrate a process flow for the creation of synthetic tumor models and their use in predicting drug/energy distribution and response to therapy, according to some example aspects;

FIG. 2 illustrates schematic top and perspective views of a panel of synthetic tumor models represented with spheres of randomly selected diameters within a known biological range, in accordance with some examples;

FIGS. 3A-3E illustrate a schematic view of a library of synthetic tumor models represented with morphologies within a known biological range, in accordance with some examples;

FIG. 4 illustrates an example synthetic tumor having characteristics associated with malignancy, in accordance with some examples;

FIG. 5 illustrates a schematic view of a library of synthetic tumor models represented with vascular distributions within a known biological range, in accordance with some examples; and

FIG. 6 illustrates a density distribution heat map in tumor images, in accordance with some examples;

FIG. 7 illustrates an example method of using the synthetic tumor model to determine predicted outcomes with respect to one or more therapeutic therapies, in accordance with some examples; and

FIG. 8 illustrates another example method of using the synthetic tumor model to determine predicted outcomes with respect to one or more therapeutic therapies, in accordance with some examples.

### DETAILED DESCRIPTION

Various embodiments of the disclosure are discussed in detail below. While specific implementations are discussed, it should be understood that this is done for illustration purposes only. A person skilled in the relevant art will recognize that other components and configurations may be used without parting from the spirit and scope of the disclosure.

Additional features and advantages of the disclosure will be set forth in the description which follows, and in part will be obvious from the description, or can be learned by practice of the herein disclosed principles. The features and advantages of the disclosure can be realized and obtained by means of the instruments and combinations particularly pointed out in the appended claims. These and other features of the disclosure will become more fully apparent from the following description and appended claims, or can be learned by the practice of the principles set forth herein.

The disclosed examples are directed to in silico systems and methods for predicting effects of any therapy or combination of therapies for the treatment of a tumor using synthetic tumor models. The in silico systems and methods may be directed at creating a library of synthetic tumor models and therapeutic response assessments from CT images via ray casting that simulates a X-ray energy beam traveling through the density values of the FEM mesh to compute a detected X-ray linear attenuation coefficient using the Beer-Lambert law equation. X-rays will be drawn at different project angles. The detections will be based on a detector configuration that defines the macro-scale configuration. The detected signal will be from a sinogram image that will be back propagated using the inverse Radon Transform through a process known as back projection of synthetic tumor models, such as those from the library of synthetic tumor models. The therapeutic response assessments may match a given patent's tumor based on its real CT images to those CT images synthesized from the library of synthetic tumor models for a determination of a probability of responses via regression (or other curve fitting/correlation) to link the CT images to emergent properties of interest, such as predicting cell death. Thus, understanding the efficacy of therapies using intravenous or intratumoral agents can be improved with the development of robust model systems to understand and predict response to the therapies.

For example, the in silico systems and methods may (1) create synthetic tumor mediums of known biological parameters at a micro-scale, (2) create a library of synthetic tumor models based on varying the biological parameters of microscale components; (3) translate the synthetic tumor models into reconstructed CT images in a macroscale, defined by a nominal CT spatial resolution encountered in clinical CT scanners, (4) determine a drug or energy distribution and response to therapy based on the reconstructed CT image representing an underlying tumoral medium, wherein prediction of therapeutic response may be based on deployment of an available therapy model that can be computed from the micro-scale representation that was used to create the library of synthetic tumor models, and (5) provide a personalized therapeutics response based on an evaluation of a given subject's CT scan from an unsupervised learned model for predicting response to therapy.

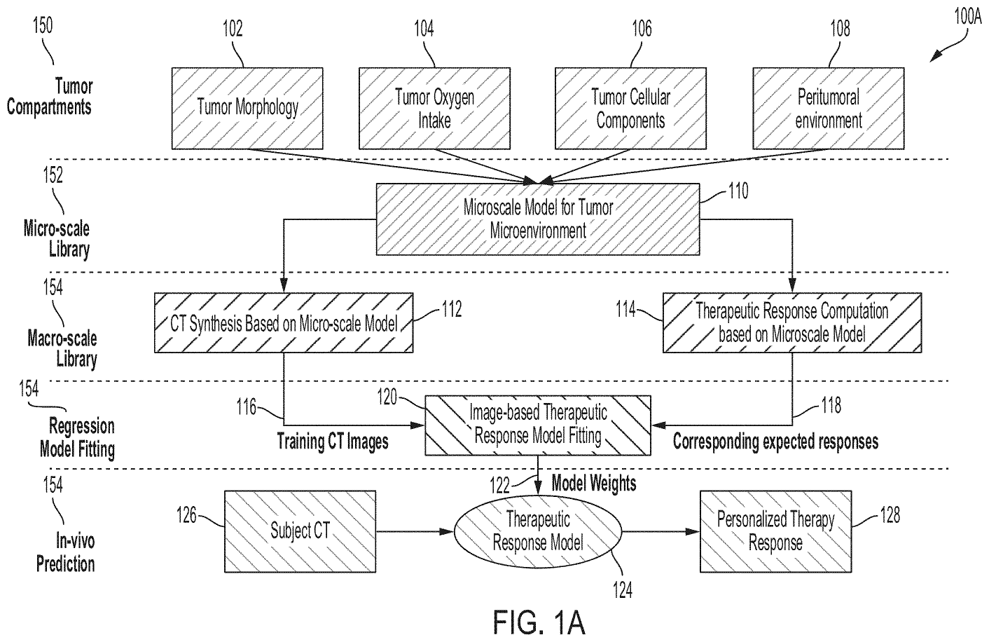

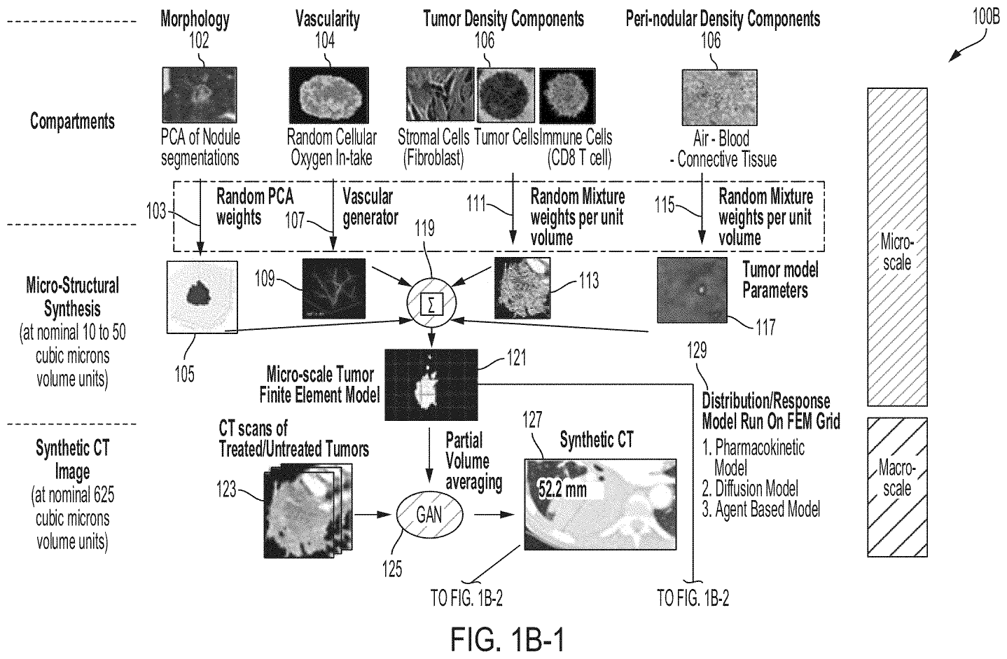

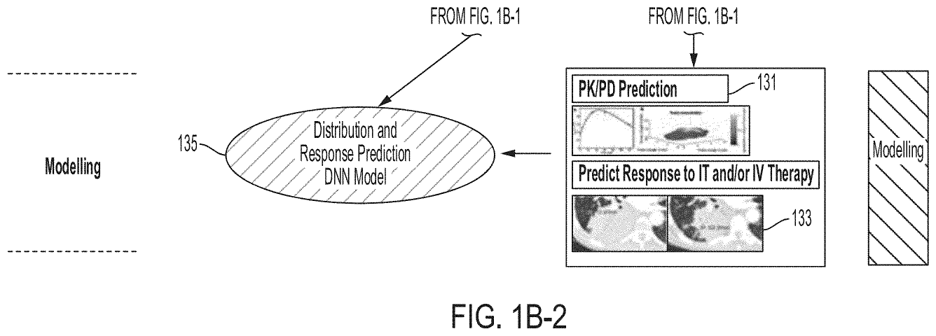

FIG. 1A illustrates a block diagram 100A of an example process of the in silico systems and methods, according to aspects of this technology. The process may include one or more functional levels, such as a tumor components analysis level 150, a micro-scale library analysis level 152, a macro-scale library analysis level 154, a regression model fitting analysis level 156, and an in vivo prediction analysis level 158, or combinations thereof, for particular needs.

With respect to the tumor components analysis level 150, analysis may be associated to parameters that define a possible tumor environment at a micro-scale. The parameters may be drawn at random to generate different instances of the synthetic tumor model. Such parameters may include a tumor morphology parameter(s) 102, a tumor oxygen intake parameter(s) 104, a tumor cellular components parameter(s) 106, and a peritumoral environment parameter(s) 108, all of which may be adjusted in modeling synthetic tumor models to have different aspects and attributes.

The tumor morphology parameter(s) 102 may be adjusted with respect to the shape of a tumoral region of the synthetic tumor. The tumor oxygen intake parameter(s) 104 may be adjusted with respect to oxygen intake for each unit of volume at the micro-scale level to grow the vascularization of the tumoral space. The tumor cellular components parameter(s) 106 may be adjusted with respect to the composition of the cellular elements of each unit of volume at the micro-scale level to define tumoral cell composition and bulk tumoral density. The peritumoral environment parameter(s) 108 may be adjusted with respect to the composition of the air-blood-tissue in the synthetic tumoral parenchyma surrounding the synthetic tumor model for each unit of volume at the micro-scale.

In the micro-scale library analysis level 152, the different tumor components of the tumor components analysis level 150 may be integrated to define and form a synthetic tumor model 110 based on data pertaining to the micro-environment of the different tumor micro-scale components based on underlying tumor component parameters. The micro-scale library of varying tumor densities, underlying cellular compositions, and tumor microenvironments may be generated by a selection of parameters set at the tumor components analysis level 150, forming the library of synthetic tumor models. The selection of the parameters may be random.

In the macro-scale library analysis level 154, synthetic CT images 112 as well as therapeutic response computations 114 may be delivered based on the synthetic tumor model 110. In the regression model fitting analysis level 156, the synthetic CT images 112 may be trained (116) and the corresponding therapeutic responses may be correlated (118), which may collectively create an image-based therapeutic response model fitting 120. Then, with the incorporation of model weights 122, the image-based therapeutic response model fitting 120 may form a therapeutic response model 124 that can be used for extrapolation of personalized therapeutic responses 128 based on in-vivo subject CT scans 126. This therapeutic response model 124 may be conditioned over time based on sampling of the tumor parameter ranges.

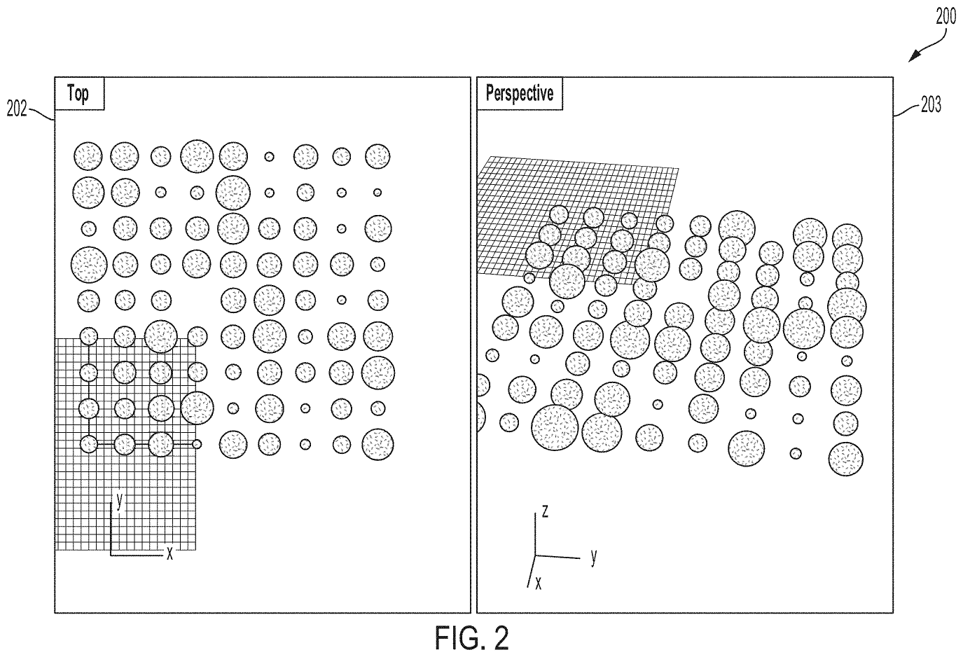

FIG. 1B-1 and 1B-2 illustrate a workflow 100B of the in silico systems and methods for creating synthetic tumor models and predicting drug distribution and response to therapy for sample tumors, according to some examples. In setting the tumor morphology parameter 102, a principal component analysis (PCA) model of nodule segmentations may be used based on CT scans of in vivo tumors. The tumor meshes may be anatomically accurate representations of the morphology of the CT-scanned in vivo tumors. As described in more detail below, by randomly altering (103) PCA weights, a morphological model 105 may be generated.

In setting the tumor oxygen intake parameter 104, cellular oxygen intake may be randomized by varying tumor vascularity, such as by using a vascular generator 107, wherein an oxygenation map may be used to create an in silico internal vascular structure model 109 of the synthetic tumor models. As described in more detail below, the oxygenation map may be used to describe cellular oxygen consumption and may be modeled to reproduce an angiogenic process that is common in tumoral regions.

In addition to varying a biologically plausible morphology and an internal vascular bed, the microcellular components of the synthetic tumor may be varied. In setting the tumor cellular components parameter 106, the in silico composition of the cellular tissue constituents (e.g., tumor cells, immune cells, stromal cells, tissue matrix, etc.) of the synthetic tumor model may be varied with respect to setting a particular structural density and pathophysiologic function based on assessment of emergent systems behavior. The different number of cells of each kind may be randomized by a random mixture weights per unit volume 111 at a micro-scale. As described in more detail below, the variance in density can be identified on a CT scan and shown in a heat map 113.

In setting the perinodular density components parameter 108, such as a structural construction of a perinodular environment, the relationship between the area surrounding the tumor and the response to therapy may also be modeled in silico. The area surrounding the tumor is known to partially determine the biological behavior and response to therapy of a tumor. Using a similar approach as discussed with reference to the synthetic tumor model 110, the perinodular vascular and structural environment of the region surrounding the synthetic tumor model 110 may be defined, wherein the proportion of each compartment for unit volume may be generated at random by a random mixture of weights per unit volume 115 and the final density may be computed as an aggregate composition model 117 based on the randomly defined proportions.

During micro-structural synthesis, such as at nominal 10 to 50 cubic microns volume units, data associated with the morphological model 105, the internal vascular structure model 109, the heat map 113, and the aggregate composition model 117 may be aggregated (119) to form a synthetic tumor model 110, such as a tumor finite element model (FEM) 121 defined over the tumor and perinodular region. As described in detail below, the generated tumor FEMs 121, or FEM meshes/grids, may be generated based on a random parameter selection at the micro-scale.

Then, back projections of the synthetic tumor model 110 or tumor FEM 121 allows creation of CT images of those synthetic tumor model 110 or tumor FEM 121 (or a subset that responds to specific therapy). While the CT images via back projections of the synthetic tumor model 110 may appear roughly similar to those of actual tumors, critical details may be lacking that may be important with respect to structure and function. Such features may be captured from CT images of actual tumors 123 and added to the synthetic tumor back projections by using a generative adversarial network (GAN) 125 to form a synthetic CT 127 image. The GAN 125 is a form of generative network that may contain the capability of creating new content derived from a series of exemplars. For example, the GAN 125 may use a deep learning algorithm to generate new samples that emanate from the synthetic CT image 127 and follows a distribution function of series of real CT tumor images.

Based on the synthetic tumor models 110 or the tumor FEMs 121, a distribution/response model 129 that runs on FEM meshes/grids may be used to predict response to various therapies. The distribution/response model 129 may be, for example, a pharmacokinetic/pharmacodynamics (PK/PD) model, a diffusion model, or an agent-based model, depending on desired outcomes. For instance, an FEM mesh allows both accurate representation of complex tumor geometry while simultaneously accounting for dissimilar tumor densities. A diffusion model may then be applied to the mesh accounting for variation in diffusivity in each of the discretized regions. When coupled with cellular uptake parameters and a third compartment encompassing blood flow, and parameterized from measurement of plasma drug levels and standard pharmacokinetic principles, a more accurate final intratumoral drug distribution may be derived. However, the endpoint may also be extended beyond understanding distribution to predicting response. In an alternative example, each cell may be treated as an “agent” with a proscribed series of properties. In this model, each tumor cell may have an assigned rate of drug uptake as determined from a pre-specified distribution (that could be assigned for example from genomic techniques including single cell RNA sequencing, tumor organoids, or other experimental systems) and a drug sensitivity (the quantity of the agent necessary to kill the cell in question) again determined from a distribution. Denser tissue areas would carry more, or additional specific types of cells, such as fibroblasts, that would modify the penetration and uptake of the drug, as well as the response of immune effector cells. Each cell of each cell type, would thus follow a pre-specified set of rules, dependent upon the interactions of other cells in the region, leading to an emergent behavior of the system, such as response to therapy. The PK/PD, agent, compartment, or diffusion predictions 131 and/or other prediction responses 133, such as to IT and/or IV therapy, may be paired with the synthetic CT images 127 to form a distribution and response prediction deep neural network (DNN) model 135.

### Creation of a Biologically Plausible Synthetic Tumor Morphology

FIG. 2 illustrates an example library 200 of simple spheres with randomly selected diameters (including a top view 202 and a perspective view 204). If the diameter were chosen as the feature to randomize, FIG. 2 shows the range of randomly selected diameters within a known biological range, such as between 2 and 8 cm, in accordance with some examples, to encapsulate in the synthetic tumor model 110. Creating spheres of known diameters serves as an example of encapsulating a feature from real tumors and then utilizing a randomization based on a known biological range to create biologically realistic synthetic tumors.

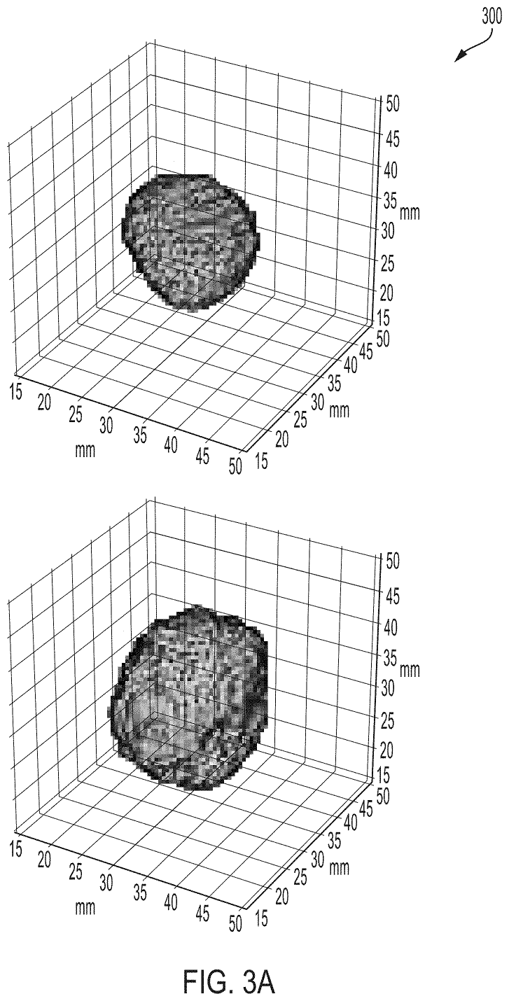

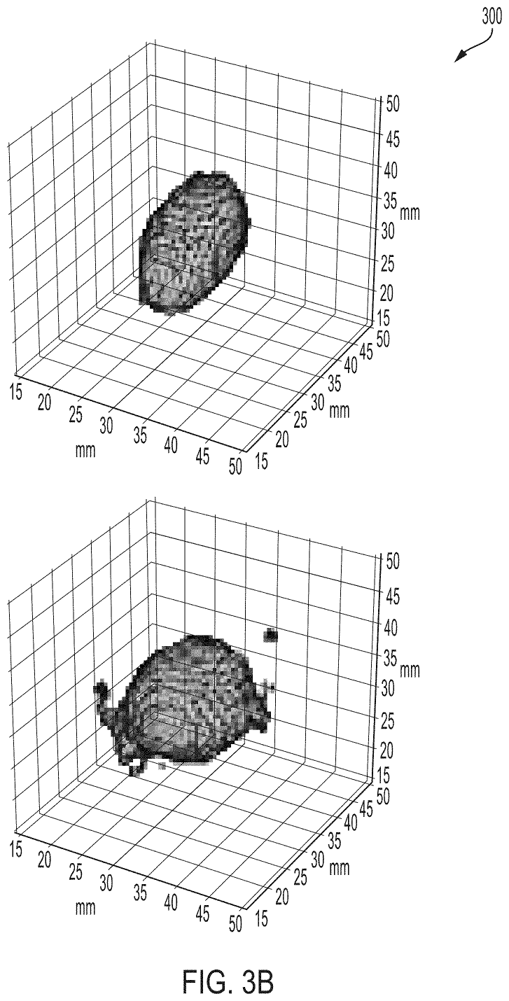

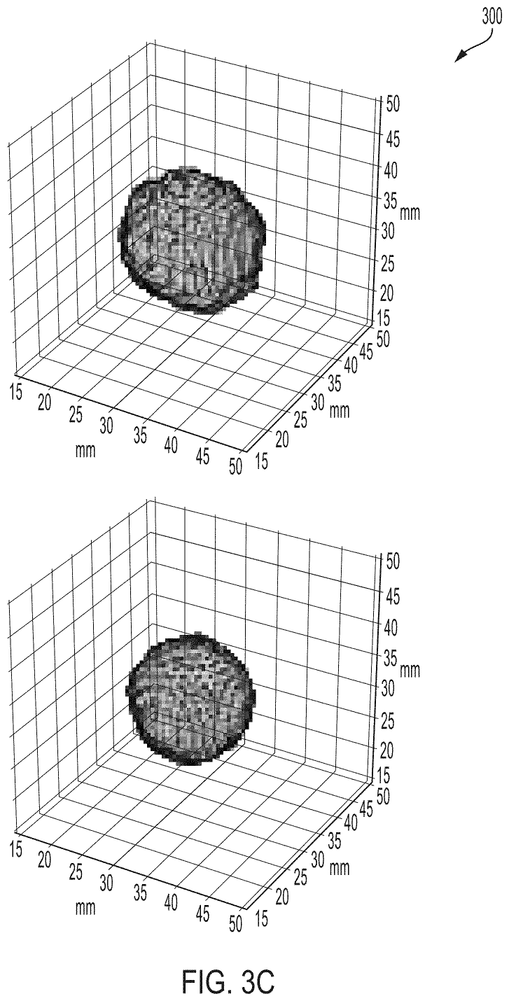

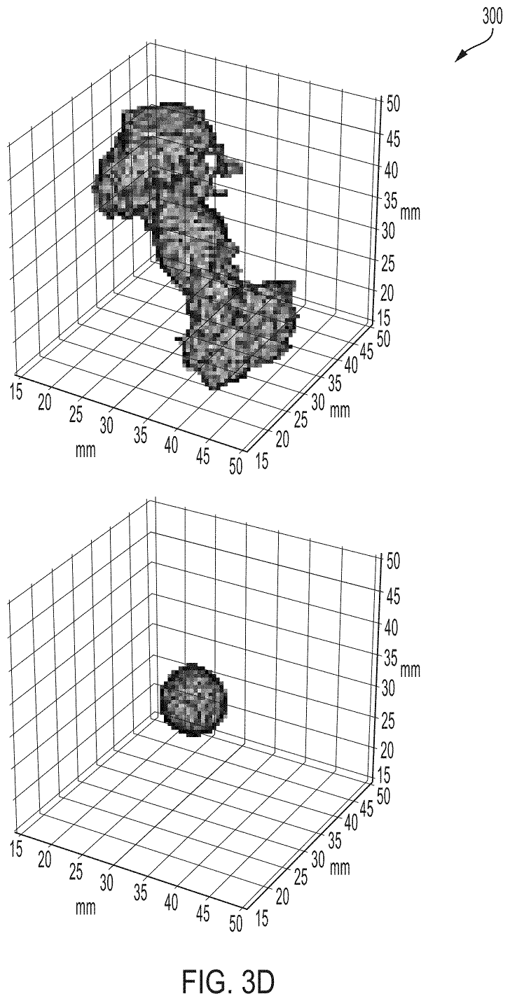

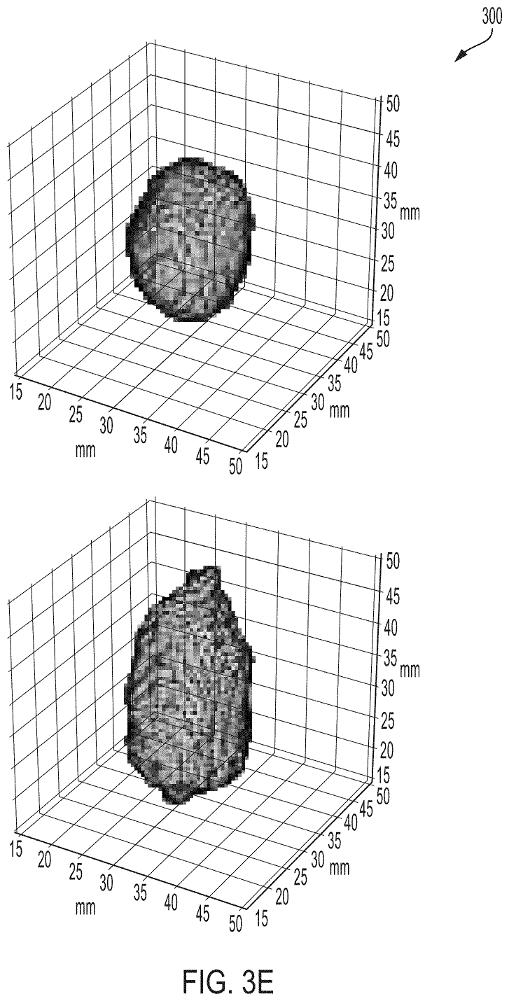

A pipeline used in the creation of the synthetic tumor models can begin with a series of in vivo tumors identified on actual CT scans. The in vivo tumors may be segmented from its surrounding structures, such as the parenchyma and mediastinum, such as by an automated or semi-automated algorithm or manual delineation to form tumor meshes as those shown in FIGS. 3A-3E. Known radiomic features may be extracted from the segmentation and aggregated using a principal components analysis (in eigen space), and the parameters for such a model may then be randomly selected. FIGS. 3A-3E illustrate a library of morphologies of synthetic tumor models 300 having model parameters randomly altered within a biologically determined range to create new morphologies. The tumor mesh, representing morphology of the scanned in vivo tumors, may then be extracted or projected to a space of fewer dimensions using a dimensionality reduction techniques, such as using principal components decomposition of distance map images using a distance transform. The distance map image may encode a distance of each voxel in the image to the closest tumor boundary element, each voxel being a unit of graphic information that defines a point in three-dimensional space. The result of the PCA decomposition may be a set of eigenbases (images) that encodes the different modes of variation of the distance map to the tumor boundary.

As mentioned previously, by randomly altering the coefficients that weight the eigen-bases of the PCA model, a new distance map that synthesizes a new tumor morphology may be generated. By thresholding the distance map at a zero-level, a binary map that defines the tumor region may be extracted and may be converted to the morphological model 105, or a tumor mesh in a 3D space. The coefficients of the PCA model may be altered within a range that is determined by the variation of the actual tumor measures defined as the square root of the eigenvalues of the PCA decomposition.

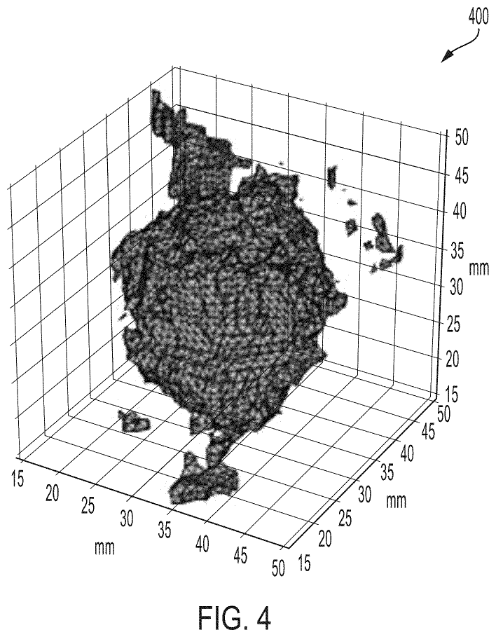

Consequently, the library of morphologies of synthetic tumor models 300 with randomly imposed variation in shape within a known biological range may be established, wherein the synthetic tumor models recapitulate biologically relevant features. Random altering as such may result in characteristics as that shown in FIG. 4, which illustrates an example morphology of a synthetic tumor model 400 having characteristics associated with malignancy. The example morphology of the synthetic tumor model 400 demonstrating a linear growth pattern wherein a part is extending out linearly, known as spiculation, is an example characteristic that is associated with malignancy.

### Development of Internal Tumor Vascularity

While an internal vascular structure of a tumor may be difficult to be readily determined from a CT scan, the biological factors that drive vascular neogenesis are well described and can be used to predict the internal vascular structure of a tumor. Therefore, a library of synthetic tumor models with known vascular distribution may provide valuable vascular analysis of CT scans. For example, a tree growth algorithm may be used to leverage how a vascular bed within the tumor “grows” based on a cellular tumor oxygen in-take environment that may form the oxygenation map. The oxygenation map may describe cellular oxygen consumption, which may be generated as a random field. The random field may be defined based on nominal values determined by metabolic activity of the cellular substrate that is being simulated.

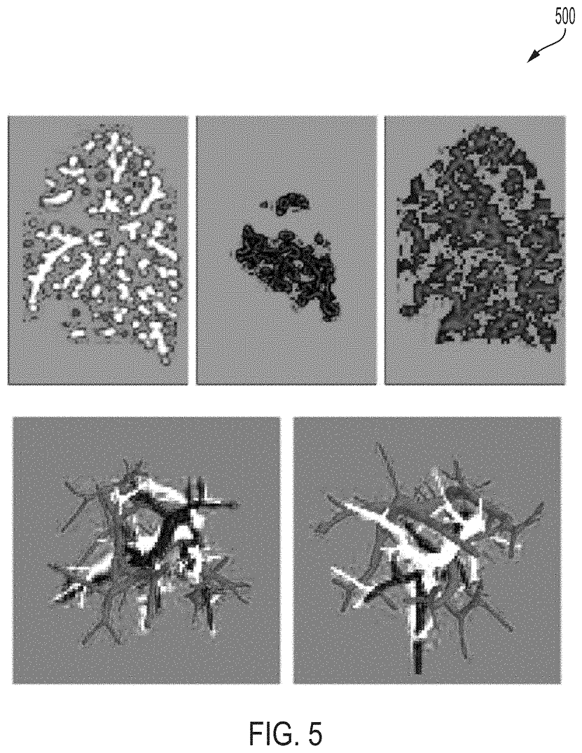

Consequently, the oxygenation map may reproduce an angiogenic process that is common in tumoral regions. FIG. 5 illustrates an example synthetic vascular model 500 of the internal vascular structure of three vascular trees grown based on three-randomly generated cellular oxygen in-take maps for an example synthetic tumor model. In other words, the example synthetic vascular model 500 may represent vascular distributions within a known biological range.

### Cellular Constituents Impart Structural Density and Pathophysiologic Function

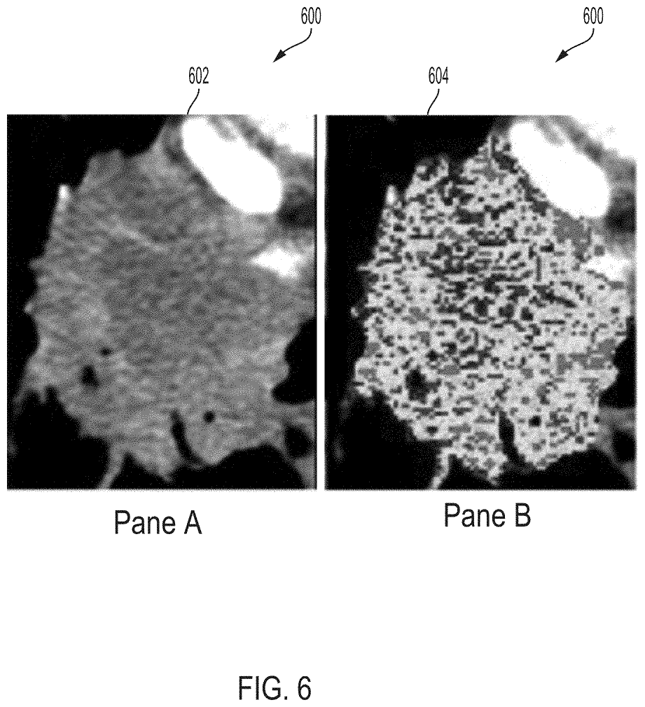

The internal structure and tissue density from the cellular constituents may be derived by populating the synthetic micro-environment with different kinds of cells, for example, stromal cells (fibroblasts), tumor cells, and immune cells. The internal structure and constituents may also be extended from a cellular level to a molecular level. The different kinds of cells and numbers of each kind within a unit volume may be randomly generated at the micro-scale level. A degree and context of a collagen mixture of the tumor may also be varied. Based on the number of cellular constitutes and a nominal cell mass, a bulk density of each unit volume may be defined based on the underlying cellular content. The variance in density may be identified in the heat map 113, which is shown in detail in FIG. 6 (Panel B) as a density distribution heat map 600 of a tumor in axial CT section (Panel A). Quantitative analysis was used to identify all voxels in the tumor via 20 Hounsfield unit (HU) density increments, where red colors represent denser voxels.

In addition, random generation of the cellular constituents and its variation may be informed by functional information. For instance, characteristics of a “hot” tumor micro-environment may be imposed by including a dense CD8+ T cell infiltrate, while incorporating tumor cells that express programmed death ligand-1 (PD-L1) may result in a tumor micro-environment with a high probability of response to anti PD-1 therapy. On the other hand, including a dense stromal environment functionally that excludes T cells may result in a “cold” immunoenvironment that would be unlikely to respond to anti PD-1 therapy. A wide range of variations can be developed with different cellular and functional characteristics for which the underlying parameters of cellular composition and bulk density will be known.

### Structural Constitution of the Perinodular Environment

The area surrounding a tumor is known to partially determine the biological behavior and response to therapy of a tumor. The relationship between the area surrounding the tumor and the response to therapy may also be modeled in silico. Using a similar approach as discussed with respect to the synthetic tumor models, the perinodular vascular and structural environment of the region surrounding the tumor. The vascular tree for the perinodular environment may use the same technique as for the bulk oxygen intake of the tumor region. The perinodular structure model may be based on a three-compartment model that includes air, blood, and connective tissue. The proportions of each component for unit volume may be generated randomly, as previously indicated, to compute an aggregate composition based on the aggregate composition model 117.

Adding to the difficulty of applying techniques, such as artificial intelligence (AI), to this problem is the biological variance of the tumors themselves. The structure of a lung tumor, for example, is highly heterogeneous, with significant variation in environmental features such as morphology, density, and vascularity, which are also referred to as macro-environmental features. Beyond these macro-environmental features, the cellular makeup of a tumor can vary not only by individual but within the tumor itself. For example, the cellular and genomic variation is observed to be greater within a tumor than between different tumors. This cellular milieu includes stromal elements, such as fibroblasts and collagen, tumor cells, and the immunocellular infiltrate. The differences in the interaction of immune cells, such as dendritic cells, T cells, and macrophages with the vascular and stromal elements, plays a critical role in the response of lung tumors to systemic (IV) immunotherapy. Moreover, regional variation in tumor macrostructure and microstructure can functionally exclude immune cells from tumor regions, resulting in resistance to immunotherapy.

### Micro-Scale Tumor Finite Element Mesh

The microstructural models of the different tumor compartments may be aggregated (119) in the synthetic tumor model 110, such as the tumor FEM 121 defined over the tumor and perinodular region. As described in detail below, each grid element may cover a unit volume in the range of micrometer range (from 5 to 50 microns) based on a desired resolution. Each grid element may include: (1) a belong state, which is a presence of either a tumor grid point or a perinodular grid point based on the morphological model 105; (2) a vascular volume, which is the amount of blood in the unit volume covered by the grid point based on the internal vascular structure model 109; (3) cell components, which is a cell count for each cell type within the unit volume based on the heat map 113; and (4) tumor density, which is a density corresponding to aggregate values of the mixture of cells and their nominal mass assigned by the aggregate composition model 117.

Critically, the synthetic tumor model 110 may be used to predict response to, for example, chemotherapy, targeted genetic therapies, and immunotherapies. For instance, an in silico assessment of an interaction of intratumoral (IT) cisplatin delivered bronchoscopically plus systemically delivered PD-1 inhibitor (immunotherapy) could be performed on a synthetic lung tumor model. Both percent tumor cytotoxic cell death and immunologic cell death could be predicted for each synthetic lung tumor model within the library. Varying numbers or distributions of cell types with differing drug combinations can then be introduced. Further, in some lung tumors the dose of cisplatin could be lethal to all the T cells within the lung tumor due to low tumor vascularity and high diffusivity in the lung tumor, with few infiltrating T cells initially and large necrotic regions. Conversely, in a lung tumor with a large subset of T cells already present and high vascularity (sweeping off more cisplatin into the systemic circulation) the combination of cisplatin to kill lung tumor cells and anti-PD-1 therapy to activate the T cells may be highly efficacious. Therefore, the synthetic tumor model 110 may be used both to assess the aggregate response of a therapy or combination of therapies within the library of synthetic tumor models (e.g. how many tumor models responded) and also to enable selection of patients likely to benefit from the therapeutic approach in question.

This methodology may also allow assessment and prediction of a number of critical features. For a systemic IV drug, for example, the library of synthetic tumor models may provide a determination of an average tumor mass of the drug following administration based on features such as tumor size and shape, tumor density (which drives diffusion of the agent), and vascular perfusion. Systemic factors such as rates of renal clearance may also be included in the model to further parameterize with respect to a pharmacokinetic/pharmacodynamic model, which may assist with more rational drug design. Drug characteristics could also be optimized to increase tumor penetrance. For example, tumors with high interstitial fluid pressures due to ongoing inflammation would harbor less systemically delivered drug due to poor migration of the drug from the vascular space into the tumor. The responsive synthetic tumor models may then undergo CT back projection to accurately identify patients who may benefit from specific therapies.

### CT Image Synthesis from Synthetic Tumor Model

The synthetic tumor models 110 or the tumor FEMs 121 recapitulate the morphology, vascular structure, and density (derived from cellular composition) of actual tumors. A CT image via back projection for the corresponding synthetic tumor model 110 may be accomplished by partial volume averaging of density values at the resolution of a CT grid, such as one order of magnitude higher at ˜0.625 mm. The averaging may be done with a Gaussian kernel to simulate the effect of the reconstruction kernel. Gaussian noise may also be introduced to simulate the quantum noise at a CT detector.

As mentioned previously, although the CT images via back projections of the synthetic tumor models 110 may appear roughly similar to actual tumors, they may lack critical details that may be important for structure and function. These features may be captured from CT images of actual tumors and added to the CT images via back projection using the GAN 125. The conversion from CT images via back projections into more realistic CT images or other reconstructed image modalities, coupled with an AI-model as trained below and specified to the outcome of interest, allows for the identification of patients who may potentially respond to a specific therapy or combination of therapies, enabling a precise approach to cancer therapeutics. For instance, a CT scan (such as a cone-beam CT scan) may be performed before and after instillation of an IV or IT agent. The changes in these images, based on simple density or textural changes at the level of the voxel for instance, may be used to “match” against back-projected CT images produced from the synthetic tumor library (e.g. CT back projection produced from the synthetic tumor(s) both before and after in silico administration of an agent). This “matching” would be performed based on prior training of a regressor, for instance a deep learning model, on the in-silico tumor library and testing on a smaller set of actual data. This regressor or model could then be applied to a set of CT images (e.g. before and after delivery of an agent) to predict the distribution of the agent.

In addition, interpolating tissue and blood vessel densities, learned from the synthetic tumor models, to CT characteristics may allow users to accurately plan where within a tumor a therapy (e.g., intratumoral chemotherapy, thermal therapies such as microwave, or cryoablation) may be applied. Using tumor characteristics available on a given CT scan, an ideal position of the delivery device (e.g., catheter, bronchoscope, transthoracic needle, etc.), an appropriate location for placement of the delivery device within the tumor (e.g., the needle), and a dose of energy or drug to be used, can be predicted, coupled with determination of the probability of response of the IT therapy, with or without additional IV therapy. Consequently, the distribution of a drug, energy, or other intratumoral therapeutic modality, and the probable therapeutic response may be based on the delivery variables and macro and micro-scale tumor characteristics.

### Synthetic Tumor Models Are Used for Prediction Drug Distribution and Therapeutic Response

With the vascular distribution for each synthetic tumor model known, a distribution of any given IV (e.g., chemotherapy, immuno-oncology agent, etc.) or intratumoral (IT) agent may be estimated. Conversely, it is difficult to strictly determine vascular flow and/or tissue distribution of tumor cells in vivo from actual tumors. Within any given synthetic tumor model, the distribution of an agent delivered either IT or IV may be determined, e.g., by numerically solving a series of partial differential equations. A distribution/response model 129 may run on the FEM 121 grid. Since we know the underlying tumor FEM 121, distribution and response outcomes may be computed on the tumor FEM space using different modeling solutions like (1) a pharmacokinetic model, (2) a diffusion model, or (3) an agent-based model (potentially derived from genomic, organoid, or other experimental data), depending on the desired outcome, which may be different depending on how the result is to be applied.

The desired outcome may be a pharmacokinetic/pharmacodynamic (PK/PD) prediction 131 that may be based on a pharmacokinetic model. The outcome may be based on any tumor characteristic, or an emergent property such as total cell death, that is the result of PK and PD cellular interactions. The desired outcome may be a response prediction 133 of certain therapies delivered, either intravenously or intratumorally, based on cellular interactions and tumor structure. The response may be produced with respect to the emergent response elements, such as number of activated T cells or dead tumor cells, based on the diffusion model.

The desired outcome may be based on specific functional responses, which may be predicted by an agent-based model. Given that the application of synthetic tumor models may extend beyond structure to function as well, responses to therapy may be predicted using the agent-based model. Each cellular component may serve as an “agent” and may be imparted with specific functions and responses. Agent-based modeling may be used for understanding complex interactions between interrelated units, or agents. Each agent may follow a set of rules, individually assesses its situation, and makes a decision based on those rules. This represents a tractable way to deal with repetitive, competitive interactions of essentially arbitrary complexity.

For example, agent rules may allow specifying how a lymphocyte, for instance, will respond to encountering a tumor cell. Extending the previous example, a tumor cell that expresses PD-L1 may be killed by the lymphocyte whereas a tumor that does not express PD-L1 would “escape.” In the same model, the effect of dense collagen areas in the tumor would exclude the T cells, providing a safe harbor for tumor cells. Complicated multimodal therapeutic scenarios may then be assessed, such as addressing the question of the potential efficacy of thermal ablation to disrupt dense collagen areas followed by IV administration of anti PD-1 therapy.

### A Deep Neural Network (DNN) is Used to Identify Patients Likely to Benefit from a Specific Therapy or Combination of Therapies

A distribution and response prediction deep neural network (DNN) model 135 may then be used to obtain a regression model based on the CT images via back projections of the synthetic tumor models. The regression model may be deployed for an evaluation of a personalized therapeutic response from a given subject's CT sample or set of CT scans. In some examples, it is also possible to utilize the given subject's CT sample in one or more of the above functional levels. For example, the subject's CT sample may be used as training data (e.g., in an AI-model, such as the GAN 125) or creating the more realistic back projection CT image. In some examples, synthetic CT images and/or the response models, etc., may be used to generate templates for finding matches amongst a collection of two or more subject CT samples.

The back projection CT images of the synthetic tumor models may serve as a large series of exemplars (and millions may be easily created) such that a set of parameters of tumor characteristics and their corresponding CT images may be generated. The distribution and response prediction DNN model 135 may be applied to actual CT images of tumors, allowing prediction of the distribution of an agent. The distribution and response prediction DNN model 135 may also allow for assessment of the distribution of energy such as radiation therapy, cryoablation, or thermal ablation.

The distribution and response prediction DNN model 135 may be a convolutional neural network or other machine learning/AI model. The outcome that is trained against may be any tumor characteristic or drug characteristic within the tumor, or an emergent property such as total cell death that is the result of pharmacokinetic and pharmacodynamics cellular interactions, and the outcome may be either continuous or discrete. The library of CT images of the synthetic tumor models may be as large as necessary to appropriately power the AI component of the distribution and response prediction DNN model 135. For instance, if the distribution and response prediction DNN model 135 contains 2 million parameters, the library of CT images may contain 4 million or more images of tumors likely to respond, providing a 2:1 ratio that results in a highly robust model. Once trained against this large number of exemplars, the distribution and response prediction DNN model 135 may then be used to provide the probability of the outcome for any given real CT image. For example, the distribution and response prediction DNN model 135 may address the likelihood of significant cell death for a given patient who was given a specified therapy. In other words, the distribution and response prediction DNN model 135 may be used to create AI-based responses models using regression techniques that match the CT images to the emergent property of interest (e.g., predicting cell death).

Furthermore, tumors with characteristics determined to be favorable for response (for example, those with more uniform tissue density, high vascular perfusion, and large numbers of infiltrating T cells) can be used to create a simulated CT scan using CT back projection. The CT characteristics of lesions may then then be used to precisely identify patients that would benefit from the therapy/therapies. Similarly, differences in registered, back-projected, synthetic CT scans can be evaluated to understand the distribution of an IV or IT agent, and used to predict a final drug distribution given an initial CT scan from a patient. This may be used for both clinical trial enrollment and personalized, precision, medicine when clinically deployed. The CT images of synthetic tumor models associated with the response outcome (e.g. cell death), vs. those that are not may be used to train a deep neural network (DNN) or another unsupervised learning model. The DNN can then be used in a new population to predict those that will respond to the therapy, how a particular individual in the new population may respond to the therapy, etc.

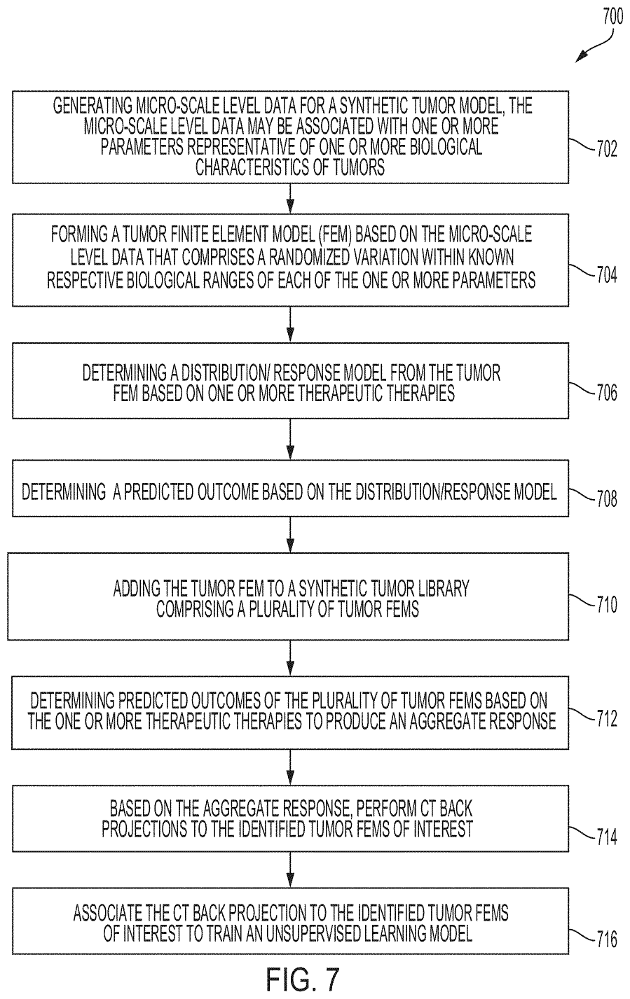

FIG. 7 illustrates an example method 700 of using the synthetic tumor model 110 to determine predicted outcomes with respect to one or more therapeutic therapies. Micro-scale data may be generated (702) for the synthetic tumor model 110 such that generated micro-scale data may be associated with one or more parameters representative of one or more biological characteristics of tumors, the micro-scale data comprising randomized variations within known respective biological ranges of each of the one or more parameters. The tumor FEM 121 may be formed (704) for the synthetic tumor model 110 based on the generated micro-scale data, such that the tumor FEM 121 may define at least one of a synthetic tumor region and a synthetic perinodular region and may be used for the therapeutic response predictions. In order to make such therapeutic response predictions, a distribution in or a response from the tumor FEM 121 may be determined (706) based on one or more therapeutic therapies, such as an energy source, an intravenous agent, and/or an intratumoral agent using known vascular flow or tissue distribution based on the micro-scale data. Consequently, a predicted outcome (708) may be determined with respect to emergent response elements, tumor characteristics, one or more emergent properties, potential efficacy, local therapeutics effects, local side effects, and/or systemic side effects. The distribution or the response may be modeled based on at least one of a pharmacokinetic/pharmacodynamic model, a diffusion model, and an agent-based model.

The tumor FEM may be added (710) to a synthetic tumor library including a plurality of tumor FEMs. Each of the plurality of tumor FEMs may define at least one of a synthetic tumor or a synthetic perinodular region or a combination thereof. Then based on the synthetic tumor library, predicted outcomes may be determined (712) for the plurality of tumor FEMs based on the one or more therapeutic therapies to produce an aggregate response. The aggregate response may include a determination of an average of a tumor mass following administration of the one or more therapeutic therapies. Then, one or more CT back projections may be performed (714) on one or more identified tumor FEMs having predicted outcomes of interest from the one or more therapies. Predicted outcomes of interest may be either advantageous (e.g., increased cell death) or disadvantageous for purposes of research and understanding the effects of the one or more therapeutic therapies.

Consequently, based on the one or more CT images via back projection and associated tumor FEMs, an unsupervised learning model may be trained (716) to distinguish between CT images of synthetic tumor models associated with responsive outcomes versus CT images of synthetic tumor models associated with less responsive outcomes. The unsupervised learning model may be a non-linear regression model encoded as a deep neural network or regression method. Furthermore, based on the unsupervised learning model, a personalized treatment plan may be determined (718) based on similar characteristics found in the one or more CT images and a CT scan of a patient tumor to determine an optimal location of placement of a delivery device within the patient tumor, and/or a distribution of a drug, energy, or other intratumoral therapeutic modality or a combination thereof within the patient tumor.

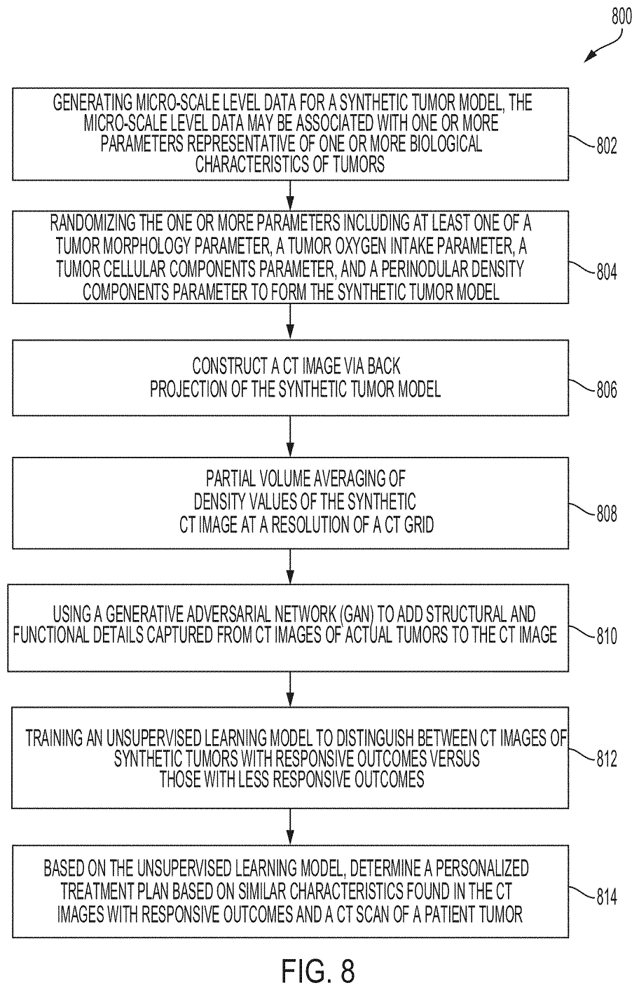

FIG. 8 illustrates another example method 800 of using the synthetic tumor model 110 to determine predicted outcomes with respect to one or more therapeutic therapies. Micro-scale data may be generated (802) for the synthetic tumor model 110 such that generated micro-scale data may be associated with one or more parameters representative of one or more biological characteristics of tumors. The micro-scale data may comprise randomized variations within known respective biological ranges of each of the one or more parameters.

The micro-scale data may be randomized (804) based on one or more parameters such as a tumor morphology parameter, a tumor oxygen intake parameter, a tumor cellular components parameter, and a perinodular density components parameter to form the synthetic tumor model 110. The tumor morphology parameter may depend on random PCA weights, the tumor oxygen intake parameter may depend on a vascular generator, the tumor cellular components parameter may depend on ratios of cellular tissue constituents per unit volume, and the perinodular density components parameter may depend on ratios of perinodular cellular tissue constituents per unit volume.

The synthetic tumor model 110 may be the tumor FEM 121 and synthetic CT images may be constructed (806) via back projection of the tumor FEM 121. Generally, the synthetic CT image may be based on an image-based therapeutic response model fitting that is trained by CT images associated with corresponding therapeutic responses. Model weight may be incorporated with the image-based therapeutic response model fitting to condition a therapeutic response model for extrapolation of personalized therapeutic responses based on in vivo subject CT scans.

More specifically, the constructing of the synthetic CT images may further comprise partial volume averaging (808) of density values of the synthetic CT projection at a resolution of a CT grid by at least one of the following: using a Gaussian kernel that simulates effects of a reconstruction kernel and introducing Gaussian noise that simulates quantum noise at a CT detector. The constructing of the synthetic CT images may further comprise using (810) a generative adversarial network (GAN) that uses a deep-learning algorithm to add structural and functional details captured from CT images of actual tumors to the synthetic tumor projection to create one or more improved synthetic CT images.

Based on the one or more improved synthetic CT images and associated tumor FEMs, an unsupervised learning model may be trained (812) to distinguish between CT images of synthetic tumor models associated with responsive outcomes versus CT images of synthetic tumor models associated with less responsive outcomes. The unsupervised learning model may be a non-linear regression model encoded as a deep neural network or regression method. Based on the unsupervised learning model, a personalized treatment plan may be determined (814) based on similar characteristics found in the one or more improved synthetic CT images and a CT scan of a patient tumor to determine an optimal location of placement of a delivery device within the patient tumor and/or a distribution of a drug, energy, or other intratumoral therapeutic modality or a combination thereof within the patient tumor.

For clarity of explanation, in some instances the present technology may be presented as including individual functional blocks including functional blocks comprising devices, device components, steps or routines in a method embodied in software, or combinations of hardware and software.

In some embodiments, the computer-readable storage devices, mediums, and memories can include a cable or wireless signal containing a bit stream and the like. However, when mentioned, non-transitory computer-readable storage media expressly exclude media such as energy, carrier signals, electromagnetic waves, and signals per se.

Methods according to the above-described examples can be implemented using computer-executable instructions that are stored or otherwise available from computer readable media. Such instructions can comprise, for example, instructions and data which cause or otherwise configure a general-purpose computer, special purpose computer, or special purpose processing device to perform a certain function or group of functions. Portions of computer resources used can be accessible over a network. The computer executable instructions may be, for example, binaries, intermediate format instructions such as assembly language, firmware, or source code. Examples of computer-readable media that may be used to store instructions, information used, and/or information created during methods according to described examples include magnetic or optical disks, flash memory, USB devices provided with non-volatile memory, networked storage devices, and so on.

Devices implementing methods according to these disclosures can comprise hardware, firmware and/or software, and can take any of a variety of form factors. Some examples of such form factors include general purpose computing devices such as servers, rack mount devices, desktop computers, laptop computers, and so on, or general purpose mobile computing devices, such as tablet computers, smart phones, personal digital assistants, wearable devices, and so on. Functionality described herein also can be embodied in peripherals or add-in cards. Such functionality can also be implemented on a circuit board among different chips or different processes executing in a single device, by way of further example.

The instructions, media for conveying such instructions, computing resources for executing them, and other structures for supporting such computing resources are means for providing the functions described in these disclosures.

Although a variety of examples and other information was used to explain aspects within the scope of the appended claims, no limitation of the claims should be implied based on particular features or arrangements in such examples, as one of ordinary skill would be able to use these examples to derive a wide variety of implementations. Further and although some subject matter may have been described in language specific to examples of structural features and/or method steps, it is to be understood that the subject matter defined in the appended claims is not necessarily limited to these described features or acts. For example, such functionality can be distributed differently or performed in components other than those identified herein. Rather, the described features and steps are disclosed as examples of components of systems and methods within the scope of the appended claims.

Claim language reciting “at least one of” a set indicates that one member of the set or multiple members of the set satisfy the claim. For example, claim language reciting “at least one of A and B” means A, B, or A and B.

What is claimed is:

1. A system of creating a synthetic tumor model for therapeutic response predictions, the system comprising:
one or more processors; and
at least one non-transitory computer-readable medium storing instructions that, when executed by the one or more processors, cause the one or more processors to:
generating micro-scale data for the synthetic tumor model, generated micro-scale data associated with one or more parameters representative of one or more biological characteristics of tumors, the micro-scale data comprising randomized variations within known respective biological ranges of each of the one or more parameters;
forming a tumor finite element model (FEM) for the synthetic tumor model based on the generated micro-scale data, the tumor FEM defining at least one of a synthetic tumor region and a synthetic perinodular region;
constructing one or more synthetic CT images via back projection of the tumor FEM by partial volume averaging of density values at a resolution of a CT grid by at least one of using a Gaussian kernel that simulates effects of a reconstruction kernel and introducing Gaussian noise that simulates quantum noise at a CT detector;
using a generative adversarial network (GAN) that uses a deep-learning algorithm to add structural and functional details captured from CT images of actual tumors to the synthetic tumor projection to create one or more improved synthetic CT images;
training, based on the one or more improved synthetic CT images and associated tumor FEMs, an unsupervised learning model to distinguish between synthetic CT images of synthetic tumor models associated with responsive outcomes versus synthetic CT images of synthetic tumor models associated with less responsive outcomes; and
determining, based on the unsupervised learning model, a personalized treatment plan based on similar characteristics, or differences in characteristics, found in the one or more improved synthetic CT back projections and a CT scan a patient tumor to determine at least one of:
an optimal location of placement of a delivery device within the patient tumor; and
a distribution of a drug, energy, or other intratumoral therapeutic modality or a combination thereof within the patient tumor.

2. The system of claim 1, wherein the micro-scale data is randomized based on one or more parameters including at least one of a tumor morphology parameter, a tumor oxygen intake parameter, a tumor cellular components parameter, and a perinodular density components parameter.

3. The system of claim 2, wherein the tumor morphology parameter depends on random PCA weights, the tumor oxygen intake parameter depends on a vascular generator, the tumor cellular components parameter depends on ratios of cellular tissue constituents per unit volume, and the perinodular density components parameter depends on ratios of perinodular cellular tissue constituents per unit volume.

4. A system of creating a synthetic tumor model for therapeutic response predictions, the system comprising:
one or more processors; and
at least one non-transitory computer-readable medium storing instructions that, when executed by the one or more processors, cause the one or more processors to:
generating micro-scale data for the synthetic tumor model, generated micro-scale data associated with one or more parameters representative of one or more biological characteristics of tumors, the micro-scale data comprising randomized variations within known respective biological ranges of each of the one or more parameters;
forming the synthetic tumor model based on the generated micro-scale data;
constructing one or more synthetic CT images via back projection of the synthetic tumor model, wherein the one or more synthetic CT images is based on an image-based therapeutic response model fitting that is trained by other CT images or differences in CT images, associated with corresponding drug distribution or therapeutic responses; and
incorporating model weights with the one or more synthetic CT images to condition a therapeutic response model for extrapolation of personalized therapeutic responses.

5. The system of claim 4, wherein the micro-scale data is randomized based on one or more parameters including at least one of a tumor morphology parameter, a tumor oxygen intake parameter, a tumor cellular components parameter, and a perinodular density components parameter.

6. The system of claim 5, wherein the tumor morphology parameter depends on a random PCA weights, the tumor oxygen intake parameter depends on a vascular generator, the tumor cellular components parameter depends on ratios of cellular tissue constituents per unit volume, and the perinodular density components parameter depends on ratios of perinodular cellular tissue constituents per unit volume.

7. The system of claim 4, wherein the instructions further cause the one or more processors to:
training, based on the one or more synthetic CT images and associated synthetic tumor models, an unsupervised learning model based on the image-based therapeutic response model fitting to distinguish between CT images of synthetic tumor models associated with responsive outcomes versus CT images of synthetic tumor models associated with less responsive outcomes.

8. The system of claim 7, wherein the unsupervised learning model is a non-linear regression model encoded as a deep neural network or regression method.

9. The system of claim 8, wherein the instructions further cause the one or more processors to:
determining, based on the unsupervised learning model, a personalized treatment plan based on similar characteristics, or differences in characteristics, found in the one or more synthetic CT images and a CT scan a patient tumor to determine at least one of:
an optimal location of placement of a delivery device within the patient tumor; and
a distribution of a drug, energy, or other intratumoral therapeutic modality or a combination thereof within the patient tumor.

10. A method of creating a synthetic tumor model for therapeutic response predictions, the method comprising:
generating micro-scale data for the synthetic tumor model, generated micro-scale data associated with one or more parameters representative of one or more biological characteristics of tumors, the micro-scale data comprising randomized variations within known respective biological ranges of each of the one or more parameters; and
forming the synthetic tumor model based on the generated micro-scale data.

11. The method of claim 10, wherein the micro-scale data is randomized based on one or more parameters including at least one of a tumor morphology parameter, a tumor oxygen intake parameter, a tumor cellular components parameter, and a perinodular density components parameter.

12. The method of claim 11,
wherein the tumor morphology parameter depends on a principal component analysis (PCA) weight for a PCA model of nodule segmentation based on CT scans of in vivo tumors,
wherein the tumor oxygen intake parameter depends on a vascular generator that varies tumor vascularity based on cellular oxygen consumption described by an oxygenation map formed by a tree growth algorithm,
wherein the tumor cellular components parameter depends on ratios of cellular tissue constituents per unit volume, and
wherein the perinodular density components parameter depends on ratios of perinodular cellular tissue constituents per unit volume.

13. The method of claim 10, further comprising:
constructing one or more synthetic CT images via back projection of the synthetic tumor model.

14. The method of claim 13, wherein the one or more synthetic CT images is based on an image-based therapeutic response model fitting that is trained by other CT images associated with corresponding therapeutic responses.

15. The method of claim 14, further comprising:
incorporating model weights with the one or more synthetic CT images to condition a therapeutic response model for extrapolation of personalized therapeutic responses based on in-vivo subject CT scans.

16. The method of claim 13, wherein the constructing the one or more synthetic CT images further comprises:
partial volume averaging of density values at a resolution of a CT grid by at least one of using a Gaussian kernel that simulates effects of a reconstruction kernel and introducing Gaussian noise that simulates quantum noise at a CT detector.

17. The method of claim 16, wherein the constructing the one or more synthetic CT images further comprises:
using a generative adversarial network (GAN) that uses a deep-learning algorithm to add structural and functional details captured from CT images of actual tumors to create one or more improved synthetic CT back projections.

18. The method of claim 17, further comprising:
training, based on the one or more improved synthetic CT back projections and associated tumor FEMs, an unsupervised learning model to distinguish between CT images of synthetic tumor models associated with responsive outcomes versus CT images of synthetic tumor models associated with less responsive outcomes.

19. The method of claim 18, wherein the unsupervised learning model is a non-linear regression model encoded as a deep neural network or regression method.

20. The method of claim 19, further comprising:
determining, based on the unsupervised learning model, a personalized treatment plan based on similar characteristics found in the one or more improved synthetic CT back projections and a CT scan a patient tumor to determine at least one of:
an optimal location of placement of a delivery device within the patient tumor; and
a distribution of a drug, energy, or other intratumoral therapeutic modality or a combination thereof within the patient tumor.
